(19) United States
(12) Patent Application Publication
(10) Pub. No.: US 2009/0130224 A1
(43) Pub. Date: May 21, 2009

(54) Granular bleach-activating mixtures

(76) Inventors: Georg Borchers, Bad Nauheim (DE); Andreas Schottstedt, Hofheim (DE); Gerd Reinhardt, Kelkheim (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/991,051

(22) PCT Filed: Aug. 25, 2006
(86) PCT No.: PCT/EP2006/008353

§ 371 Date: Apr. 9, 2008

(30) Foreign Application Priority Data
Sep. 3, 2005 (DE) 10 2005 041 967.4

(51) Int. Cl. A61K 33/40; C11D 3/395; C11D 3/39; A01P 1/00; A61K 47/12

(52) U.S. Cl. 424/616; 510/513; 514/784; 510/375

(57) ABSTRACT

The invention relates to an improved bleach activator and bleach activator compositions in the form of granules for use in washing, cleaning and disinfectant compositions. More specifically, the invention relates to granular bleach-activating mixtures that consist substantially of a) a hydroxybenzoic acid derivative of formula (I)

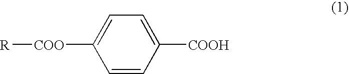

in which R is C₈-C₁₁-alkyl and b) tetraacetyl ethylene diamine and/or 1,5-diacetyl-2,4-dioxo-1,3,5-hexahydrotriazine, the granules being free of binding agents.

The present invention is described in the German priority application No. ______, filed ______, which is hereby incorporated by reference as is fully disclosed herein.

The invention relates to improved bleach activator and bleach compositions in the form of granules for use in washing, cleaning and disinfectant compositions. In particular, the invention relates to a process for granulating bleach activator cogranules comprising up to 100% active substance with good storage stability and improved bleaching performance on a multitude of bleachable stains.

Inorganic peroxygen compounds, especially hydrogen peroxide and solid peroxygen compounds which dissolve with release of hydrogen peroxide in water, such as sodium perborate and sodium carbonate perhydrate, have been used for some time as oxidizing agents for disinfection and bleaching purposes. The oxidizing action of these substances in dilute solutions depends greatly on the temperature; for example, with H₂O₂ or perborate in alkaline bleaching liquors, sufficiently rapid bleaching of soiled textiles is achieved only at temperatures above about 80° C.

At lower temperatures, the oxidizing action of the inorganic peroxygen compounds can be improved by adding so-called bleach activators. For this purpose, numerous proposals have been developed in the past, in particular from the substance classes of the N- or O-acyl compounds, for example polyacylated alkylenediamines, especially tetraacetylethylene-diamine and tetraacetylglycoluril, N-acylated hydantoins, hydrazides, triazoles, hydrotriazines, urazoles, diketopiperazines, sulfurylamides and cyanurates, and also carboxylic anhydrides, especially phthalic anhydride and substituted maleic anhydrides, carboxylic esters, especially sodium acetoxybenzenesulfonate, sodium benzoyloxybenzenesulfonate (BOBS), sodium nonanoyloxybenzenesulfonate (NOBS), sodium isononanoyloxy-benzenesulfonate (ISONOBS), and acylated sugar derivatives such as pentaacetylglucose. Addition of these substances allows the bleaching action of aqueous peroxide solutions to be enhanced to such an extent that essentially the same effects as with the peroxide solution alone at 95° C. occur even at temperatures of around 40-60° C.

Bleach activators can be divided into two classes with regard to their reactivity toward particular stains, hydrophilic and hydrophobic. Hydrophilic bleach activators remove especially tea or red wine stains, while hydrophobic activators preferably decolorize oily discolorations such as ketchup and barbecue sauce. However, many stains which occur in daily life do not fall within these classes (for example grass, curry) or are mixtures of different types of stains (for example baby food). Here, the use of a single bleach activator usually leads to unsatisfactory results. With regard to further reduced washing temperatures and more volume-efficient formulations, synergistically active mixtures of washing composition ingredients will be of particular interest in the future.

The use of specific activator mixtures consisting of a hydrophilic activator and a hydrophobic activator is prior art. The hydrophobic components used are predominantly derivatives of the readily water-soluble sodium phenolsulfonate. For example, EP-A-0 257 700 claims mixtures of nonanoyloxybenzenesulfonate with tetraacetylethylenediamine, benzoyl-oxybenzenesulfonate or acetoxybenzenesulfonate. WO 02/083 829 describes improved effectiveness of mixtures consisting of tetraacetyl-ethylenediamine and sodium (4-sulfophenyloctyl)carbonate. Similar mixtures are also described in EP-A-098 129 and EP-A-0 120 591.

According to DE 10 2004 043 360, synergistic bleaching effects on difficult-to-remove stains such as grass or curry can be achieved by mixtures of bleach activators based on hydroxybenzoic acids and derivatives thereof, preferably nonanoyloxybenzoic acid and decanoyloxybenzoic acid, and particular peracetic acid-releasing activators, preferably tetraacetyl-ethylenediamine and/or 1,5-diacetyl-2,4-dioxo-1,3,5-hexahydrotriazine. Where granules are described in this application, they always comprise a binder.

Corresponding acyloxybenzoic acids are described, for example, in EP-A-0 337 264 and DOS 196 54 780, tetraacetylethylenediamine in GB 907,356, and 1,5-diacetyl-2,4-dioxo-1,3,5-hexahydrotriazine in DD 229 696 and DD 259 634.

In a preferred embodiment, these activator mixtures are used in the form of cogranules as a bleach component together with a hydrogen peroxide-generating substance in washing, cleaning and disinfectant compositions. As well as by the water solubility of the activator and type and reactivity of the peracid formed, the achievable bleaching result is determined by the stability of the granules and by the concentration of the activator mixtures in the granule.

For the granulation of bleach activators, numerous granulating aids and processes have been described in the past.

EP-A-0 037 026 describes a process for producing a readily soluble activator granule having active contents between 90 to 98% by weight. To this end, the pulverulent bleach activator is mixed homogeneously with cellulose ethers or starch ethers likewise in pulverulent form and then sprayed with water or an aqueous solution of the cellulose ether or starch ether, granulated simultaneously and then dried. Since starch and cellulose derivatives form only gelatin with water, whose flowability and adhesion is insufficient, the activator granules produced by the process described in EP-A-0 037 026 have only moderate strength.

EP-A-0 240 057 and EP-A-0 241 962 describe the use of readily water-soluble film-forming polymers as binders in activator granules. Further constituents of the granules described are salts and optionally bentonite. The granules described are found to be very brittle and to have low abrasion resistance.

Generally, the question posed in granule development is that of a favorable formulation and the optimal selection of formulation constituents. What is important here is in particular the question as to the optimal binder which is needed to form the grains. This binder must not have any incompatibility with the active substance and must not impair the performance properties of the product (for example reduced solubility as a result of precipitation, or the like). Incompatibilities of the binder with other washing composition ingredients are undesired. Typically, the most suitable binder can be selected only experimentally, which in many cases requires laborious and complicated test series. To simplify the development process and granule formulation, it is therefore desirable to produce bleach activator granules as far as possible without binder. In the granulation, preference is given to working only with water as an assistant, which can be removed virtually completely from the granule in the course of subsequent drying.

Granulating aids and carrier substances additionally frequently bring the disadvantage that they remain partly on the laundry even after the rinse operation and cause a spotty appearance.

It has been found that, surprisingly, the granules described in DE 10 2004 043 360 can also be produced without use of a binder.

The invention provides granular bleach activator mixtures consisting essentially of
a) a hydroxybenzoic acid derivative of the formula 1

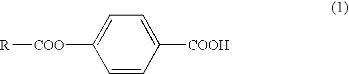

in which R is C₈-C₁₁-alkyl, and
b) tetraacetylethylenediamine and/or 1,5-diacetyl-2,4-dioxo-1,3,5-hexa-hydrotriazine, said granules being free of binders.

Preference is given to granules comprising
a) nonanoyloxybenzoic acid and decanoyloxybenzoic acid and
b) tetraacetylethylenediamine and/or 1,5-diacetyl-2,4-dioxo-1,3,5-hexa-hydrotriazine.

The compound of the formula (I) can be processed in anhydrous form or with a water content up to 50% by weight. In the latter case, the energy intensity to produce a dry product can be dispensed with. In that case, what is necessary is merely the otherwise customary final drying of the finished granule. According to the reason for final drying, granules with an activator content up to 100% by weight can be produced.

In preferred embodiments, the mixing ratio of the bleach activators hydroxybenzoic acid derivative to N-acyl compound in the granule is in the range from 95:5 to 5:95% by weight, preferably from 75:25 to 25:75% by weight, but especially from 60:40 to 40:60% by weight.

The inventive bleach activator granules comprise the bleach activator mixture in amounts by weight of from 95% to 100%, preferably from 97% to 99%, based on the granule. The residual content is water.

In further preferred embodiments, the inventive bleach activator granules comprising a hydroxybenzoic acid derivative and an N-acyl compound may comprise further additives excluding binders.

Useful additives include phosphonic acids and salts thereof, and acidic additives which influence the pH during storage and use. Further additives may be complexing agents and transition metal complexes, for example iron-, cobalt- or manganese-containing metal complexes, graying inhibitors, soil release polymers, dye fixatives, dye transfer inhibitors, optical brighteners or enzymes.

Suitable phosphonic acids are polyphosphonic acids, especially amino-tris(methylenephosphonic acid), ethylened iaminetetrakis(methylene-phosphonic acid) and 1-hydroxyethane-1,1-diphosphonic acid, and also salts thereof.

The acidic additives used may be sulfuric acid, sodium hydrogensulfate, phosphoric acid, sodium hydrogenphosphate, phosphonic acids and salts thereof, carboxylic acids or salts thereof, such as citric acid in anhydrous or hydrated form, glycolic acid, succinic acid, succinic anhydride, glutaric acid, glutaric anhydride, adipic acid, adipic anhydride, maleic acid, maleic anhydride or lactic acid, but also acidic polymers. Particularly suitable acidic additives are polyacrylic acid, polymaleic acid or copolymers of acrylic acid and maleic acid (Sokalan® types).

The amount of acidic additive is such that the proportion of the acidic additive in the finished granule is from about 0 to 30% by weight, preferably from 1 to 20% by weight, especially from 10 to 18% by weight.

Soil release polymers are preferably oligoesters comprising dicarboxylic acid units and diol units (glycol, alkylglycol and/or polyol units, especially polyalkylene polyglycol units. These oligoesters are preferably obtained by polycondensation of one or more aromatic dicarboxylic acids or esters thereof with diols, for example ethylene glycol and/or polyols. Optionally, these esters may also comprise polyethylene glycol, polypropylene glycol, sulfoisophthalic acid, sulfobenzoic acid, isethionic acid, C₁-C₄-alcohols, oxyalkylated C₁-C₂₄-alcohols, oxyalkylated C₆-C₁₈-alkylphenols and/or oxyalkylated C₈-C₂₄-alkylamines as monomers. To prepare the oligoesters, suitable examples of dicarboxylic acid units are terephthalic acid, phthalic acid, isophthalic acid, and the mono- and dialkyl esters with C₁-C₆-alcohols such as dimethyl terephthalate, diethyl terephthalate and di-n-propyl terephthalate, but also oxalic acid, succinic acid, glutaric acid, adipic acid, fumaric acid, maleic acid, itaconic acid, and also the mono- and dialkyl esters of carboxylic acids with C₁-C₆-alcohols, for example diethyl oxalate, diethyl succinate, diethyl glutarate, methyl adipate, diethyl-adipate, di-n-butyl adipate, ethyl fumarate and dimethyl maleate, and also dicarboxylic anhydrides such as maleic anhydride, phthalic anhydride or succinic anhydride. Preferred polyol units are polyethylene glycols having molar masses of from 500 to 5000, preferably from 1000 to 3000. In addition, SRPs comprise, as a further component, water-soluble addition products of from 5 to 80 mol of at least one alkylene oxide onto 1 mol of C₁-C₂₄-alcohols, C₆-C₁₈-alkylphenols or C₈-C₂₄-alkylamines. Preference is given to monomethyl ethers of polyethylene glycols.

Suitable alcohols which are alkoxylated are, for example, octyl alcohol, decyl alcohol, lauryl alcohol, myristyl alcohol or stearyl alcohol, but especially methanol, and also the alcohols which are obtainable by the Ziegler process and have from 8 to 24 carbon atoms or the corresponding oxo alcohols. Among the alkylphenols, especially octylphenol, nonylphenol and dodecylphenol are of significance. Among the useful alkylamines, especially the C₁₂-C₁₈-monoalkylamines are used.

Examples of useful polyols include pentaerythritol, trimethylolethane, trimethylolpropane, 1,2,3-hexanetriole, sorbitol, mannitol and glycerol.

Further additives in the inventive bleach activator granules may be dye fixatives, for example dye fixatives which are obtained by reacting diethylenetriamine, dicyandiamide and amidosulfuric acid, amines with epichlorohydrin, for example dimethylaminopropylamine and epichloro-hydrin or dimethylamine and epichlorohydrin or dicyandiamide, formaldehyde and ammonium chloride, or dicyandiamide, ethylenediamine and formaldehyde or cyanamide with amines and formaldehyde, or polyamines with cyanamides and amidosulfuric acid or cyanamides with aldehydes and ammonium salts, but also polyamine N-oxides, for instance poly(4-vinylpyridine N-oxide), e.g. Chromabond S-400 from ISP; polyvinyl-pyrrolidone, e.g. Sokalan HP 50/BASF, and copolymers of N-vinyl-pyrrolidone with N-vinylimidazole and optionally other monomers.

Dye transfer inhibitors are also useful, for example polyamine N-oxides, for instance poly(4-vinylpyridine N-oxide), e.g. Chromabond S-400 from ISP; polyvinylpyrrolidone, e.g. Sokalan HP 50/BASF, and copolymers of N-vinyl-pyrrolidone with N-vinylimidazole and optionally other monomers.

The graying inhibitors used may be carboxymethylcellulose, methyl-cellulose, hydroxyalkylcellulose, methylhydroxyethylcellulose, methyl-hydroxypropylcellulose, methylcarboxymethylcellulose and polyvinyl-pyrrolidone.

Useful enzymes may be those from the classes of the proteases, lipases, amylases, pullanases, cutinases, and cellulases, peroxidases or mixtures thereof. Useful proteases include BLAP®, Opticlean®, Maxacal®, Maxaperm®, Esperase®, Savinase®, Purafect®, Oxp and/or Duraxym®; useful amylases include Termamyl®, Amylase-LT®, Maxamyl®, Duramyl® and/or Pruafect® OxAm; useful lipases include Lipolase®, Lipomax®, Lumafast® and/or Lipozym®.

Suitable optical brighteners include all known optical brighteners, as described in “The Product and Application of Fluorescent Brightening Agents”, M. Zahradnik, Verlag Hohn Wiley & Sons, New York (1982) and in Ullmann's Encyclopedia of Industrial Chemistry, “Optical Brighteners”, A. E. Siegrist, Eckhardt, J. Kaschig, E. Schmidt, Vol. A18, Verlag VCH Publishers, New York (1991), pp. 153-176 CC.

Optical brighteners used with preference include cyclic hydrocarbons such as distyrylbenzenes, distyrylbiphenyls, diphenylstilbenes, triazinylamino-stilbenes, stilbenzyl-2H-triazoles, for example stilbenzyl-2H-naphthol-[1,2-d]triazoles and bis(1,2,3-triazol-2-yl)stilbenes, benzoxazoles, for example stilbenzylbenzoxazole and bis(benzoxazole), furans, benzofurans and benzimidazoles, for example bis(benzo[b]furan-2-yl)biphenyl and cationic benzimidazoles, 1,3-diphenyl-2-pyrazoline, coumarin, naphthal-imides, 1,3,5-2-yl derivatives, methinecyanine and dibenzothiophene 5,5-oxide.

Preference is given to anionic optical brighteners, especially sulfonated compounds.

Additionally useful are triazinylaminostilbenes, distyrylbiphenyls and mixtures thereof, 2-(4-styrylphenyl)-2H-naphtho[1,2-d]triazole, 4,4′-Bis-(1,2,3-triazol-2-yl)stilbene, aminocoumarin, 4-methyl-7-ethylamino-coumarin, 1,2-bis(benzimidazol-2-yl)ethylene, 1,3-diphenylpyrazoline, 2,5-Bis(benzooxazol-2-yl)thiophene, 2-styryl-naphtho[1,2-d]oxazole, 2-(4-styryl-3-sulfophenyl)-2H-naphtho[1,2-d]triazole and 2-(stilben-4-yl)-2H-naphthol[1,2-d]triazole.

The inventive bleach activator granules may comprise optical brighteners in amounts of from 0.001% by weight to 2% by weight, preferably from 0.002% by weight to 0.8% by weight, more preferably from 0.003% by weight to 0.4% by weight.

In addition, the inventive granular bleach activators may also comprise further suitable additives, such as anionic and nonionic surfactants which contribute to more rapid dissolution of the inventive granules. Preferred anionic surfactants are alkali metal salts, ammonium salts, amine salts and salts of amino alcohols of the following compounds: alkyl sulfates, alkyl ether sulfates, alkylamide sulfates and ether sulfates, alkylaryl polyether sulfates, monoglyceride sulfates, alkylsulfonates, alkylamide sulfonates, alkylarylsulfonates, α-olefinsulfonates, alkyl sulfosuccinates, alkyl ether sulfosuccinates, alkylamide sulfosuccinates, alkyl sulfoacetates, alkyl poly-glyceryl carboxylates, alkyl phosphates, alkyl ether phosphates, alkyl sarcosinates, alkyl polypeptidates, alkylamido polypeptidates, alkyl isethionates, alkyl taurates, alkyl polyglycol ether carboxylic acids or fatty acids such as oleic acid, ricinoleic acid, paimitic acid, stearic acid, copra oil acid salt or hydrogenated copra oil acid salts. The alkyl radical of all these compounds normally contains from 8 to 32 and preferably from 8 to 22 carbon atoms.

Preferred nonionic surfactants are polyethoxylated, polypropoxylated or polyglycerylated ethers of fatty alcohols, polyethoxylated, polypropoxylated and polyglycerylated fatty acid esters, polyethoxylated esters of fatty acids and of sorbitol, polyethoxylated or polyglycerylated fatty amides.

Further possible additives are substances which, in the wash liquor, react with the peroxycarboxylic acid released by the activator to form reactive intermediates, such as dioxiranes or oxaziridines, and can increase the reactivity in this way. Corresponding compounds are ketones and sulfonimines according to U.S. Pat. No. 3,822,114 and EP-A-0 446 982.

The amount of the additive is guided in particular by its type. For instance, acidifying additives and organic catalysts are added to enhance the performance of the peracid in amounts of from 0 to 20% by weight, especially in amounts of from 1 to 10% by weight; based on the total weight of the granules, but metal complexes are used in concentrations in the ppm range.

The inventive granular bleach activator mixtures are used with washing, cleaning and disinfectant compositions in combination with hydrogen peroxide or inorganic peroxygen compounds. Useful compounds here are primarily all alkali metal perborates, preferably in the form of the mono- or tetrahydrates, and/or alkali metal percarbonates, sodium being the preferred alkali metal. The ratio of bleach activator mixture and peroxide compound is from 1:0.5 to 1:20 parts by weight, preferably from 1:1 to 1:5 parts by weight.

The bleach activator mixtures are used in the inventive washing compositions or if the cleaning compositions are machine dishwasher detergents in concentrations of 0.1-15% by weight, preferably 1-8% by weight. In stain removal salts or disinfectants, the proportion of the bleach activator mixture may also be up to 50%.

In addition, such washing compositions, cleaning compositions and disinfectants may comprise oxidizing agents on an organic basis in the concentration range of 1-20%. These include all known peroxycarboxylic acids, for example monoperoxyphthalic acid, dodecanediperoxy acid, but in particular phthalimidoperoxycarboxylic acids (PAP).

The term “bleaching” is understood here to mean both bleaching of soil present on the textile surface and the bleaching of soil which has been detached from the textile surface and is present in the wash liquor. For bleaching of stains present on hard surfaces, the same applies mutatis mutandis. Further potential uses are in the personal care sector, for example in the bleaching of hair and for improving the effectiveness of denture cleaners. In addition, the inventive mixtures find use in commercial laundries, in wood and paper bleaching, the bleaching of cotton and in disinfectants.

The invention further relates to a process for cleaning textiles and also hard surfaces, especially of dishware, using the bleach activator mixtures mentioned in combination with the peroxide compound in aqueous solution optionally comprising further washing or cleaning composition constituents, and to washing compositions and cleaning compositions for hard surfaces, especially cleaning compositions for dishware, preference being given to those for use in machine processes.

The granulation of the bleach activator mixtures can be effected in customary batchwise or continuous mixing apparatus which are generally equipped with rotating mixer units. The mixers used may be moderate apparatus, for example plowshare mixers (Lödige KM types, Drais K-T types), but also intensive mixers (for example Eirich, Schugi, Lödige CB types, Drais K-TT types). In the mixing, all mixing variants which ensure sufficient mixing of the components are conceivable.

In a preferred embodiment, all components are mixed simultaneously.

In a further preferred embodiment, the bleach activator of the formula (I) having a water content of from 10 to 50% by weight, preferably from 20 to 40% by weight, more preferably from 25 to 35% by weight, is initially charged in a turbulent mixer or intensive mixer, and the second bleach activator is introduced and homogenized without further additives.

According to the requirements, the addition of additional amounts of water and/or exact process control to minimize water losses may be required in the course of the process.

The residence times in the mixer granulation are preferably from 0.5 s to 20 min, more preferably from 2 s to 10 min.

If required, the granulation stage is followed by a drying step in order to prevent conglutination of the granules. The aftertreatment preferably takes place in a fluidized bed apparatus. Subsequently, the coarse fraction and the fine fraction are removed by screening. The coarse fraction is comminuted by grinding and, just like the fine fraction, sent to another granulation process.

In a further preferred embodiment, the bleach activator of the formula (I) having a water content of from 10 to 50% by weight, preferably from 20 to 40% by weight, more preferably from 25 to 35% by weight is initially charged in a turbulent mixer or intensive mixer, and the second bleach activator and also further solid, molten or liquid additives are introduced and homogenized.

According to the invention, in a particularly preferred embodiment, the moist bleach activator of the formula (I) having a water content of from 10 to 40% by weight, preferably from 25 to 35% by weight, is mixed with the second pulverulent bleach activator mixture and optionally the further additives, so as to form a plastically deformable composition. The mixing step can be effected in the abovementioned mixing apparatus, but kneaders or specific extruder types (e.g. Extrud-o-mix from Hosokawa-Bepex Corp.) are also conceivable. The granulation mixture is subsequently pressed by means of tools through the die bores of a compression die, so as to form cylindrically shaped extrudates. Suitable apparatus for the extrusion process is edge-runner presses (for example from Schlüter), flat die presses (for example from Amandus-Kahl) and extruders, designed as a single-shaft machine (for example from Hosokawa-Bepex, Fuji-Paudal) or preferably as a twin-screw extruder (for example from Handle). The selection of the diameter of the die bore is dependent upon the individual case and is typically in the range of 0.7-4 mm.

The emerging extrudates have to be comminuted to the desired length and particle size by a further processing step. In many cases, a length/diameter ratio of L/D=1 is desired. In the case of cylindrical granules, the particle diameter is between 0.2 mm and 2 mm, preferably between 0.5 mm and 0.8 mm; the particle length is in the range from 0.5 mm to 3.5 mm, ideally between 0.9 mm and 2.5 mm. The length and size of the granules can be established, for example, by means of fixed stripping knives, rotating cutting knives, cutting wires or cutting blades. To round off the cut edges, the granule can subsequently be rounded once again in a rounder (for example from Glatt, Schlüter, Fuji-Paudal).

In another preferred embodiment, the extrudate is only Coarsely precrushed, and the extrudate strands are transferred directly into a rounder. The further granule shaping (cylindrical to spherical particles are possible) is effected in the rounding step; in a preferred embodiment, the process is performed in cascade operation. The size and shape of the particles can be influenced and brought about by virtue of several parameters in the rounding process. The shaping process is determined by the fill amount, the temperature of the mixture, the residence time of the mixture in the rounder, by the rotational speed of the rounding disk, and by the plastic deformability of the mixture. With decreasing plasticity, longer granules are initially obtained; in the case of yet a further decrease in the plasticity, the dust fraction increases greatly and controlled particle shaping can no longer be achieved.

After the size of the granules has been established, a final consolidation step may be required, in which water is removed. Typically, this step is performed in a fluidized bed apparatus which is operated as a dryer. Subsequently, screening removes the coarse fraction and the fine fraction. The coarse fraction is comminuted by grinding and, just like the fine fraction, fed to another granulation process.

### Compaction

In a further preferred embodiment, the moist bleach activator of the formula (I) having a water content of from 10 to 40% by weight, preferably from 25 to 35% by weight, is mixed with the second pulverulent bleach activator and optionally the further additives, and this mixture is compacted, then ground and subsequently optionally screened into individual particle fractions.

The compaction is preferably performed on so-called roll compacters (for example from Hosokawa-Bepex, Alexanderwerk, Köppern). The selection of the roller profile allows, firstly, pellets or briquettes, and, secondly, pressed slugs to be obtained. While the pressings in piece form typically only have to be removed from the fines, the slugs have to be ground to the desired particle size in a mill. Typically, the mill types used are preferably gentle milling apparatus, for example screening and hammer mills (for example from Hosokawa-Alpine, Hosokawa-Bepex) or roll mills (for example from Bauermeister, Bühler).

The fine fraction and, if appropriate, the coarse fraction are removed by screening from the granule thus obtained. The coarse fraction is fed back to the mill; the fine fraction is fed back to the compaction. For classification of the granules, it is possible, for example, to use screening machines from Aligaier, Sweco, Rhewum.

### Coating:

The granules obtained in accordance with the invention are suitable directly for use in washing and cleaning compositions. In a particularly preferred use form, they can, however, be provided with a coating by processes known per se. For this purpose, the granule is enveloped with a film-forming substance in an additional step, which can considerably influence the product properties.

Suitable coating agents are all film-forming substances, such as waxes, silicones, fatty acids, fatty alcohols, soaps, anionic surfactants, nonionic surfactants, cationic surfactants, anionic and cationic polymers, and polyalkylene glycols. Preference is given to using coating substances having a melting point of 30-100° C. Examples thereof are C₈-C₃₁ fatty acids, for example lauric acid, myristic acid, stearic acid); C₈-C₃₁ fatty alcohols; polyethylene glycols having a molar mass of from 1000 to 50 000 g/mol; fatty alcohol polyalkoxylates with from 1 to 100 moles of EO; alkanesulfonates, alkylbenzenesulfonates, α-olefinsulfonates, alkyl sulfates, alkyl ether sulfates having C₈-C₃₁ hydrocarbon radicals, polymers, for example polyvinyl alcohols, waxes, for example montan waxes, paraffin waxes, ester waxes, polyolefin waxes, silicones.

It is additionally possible for substances which do not soften or melt in the range from 30 to 100° C. to be present in dissolved or suspended form in the coating substance which does soften or melt in this range, for example homopolymers, copolymers or graft copolymers of unsaturated carboxylic acids and/or sulfonic acids and their alkali metal salts, cellulose ethers, starch, starch ethers, polyvinylpyrrolidone; mono- and polybasic carboxylic acids, hydroxycarboxylic acids or ethercarboxylic acids having from 3 to 8 carbon atoms and their salts; silicates, carbonates, bicarbonates, sulfates, phosphates, phosphonates.

Depending on the desired properties of the coated granule, the proportion of envelope substance may be from 1 to 30% by weight, preferably from 5 to 15% by weight, based on the coated granule.

For the application of the envelope substances, it is possible to use mixers (mechanically induced fluidized bed) and fluidized bed apparatus (pneumatically induced fluidized bed). Possible mixers are, for example, plowshare mixers (continuous and batchwise), ring layer mixers or else Schugi mixers. When a mixer is used, the heat treatment can be effected in a granule preheater and/or directly in the mixer and/or in a fluidized bed downstream of the mixer. To cool the coated granule, granule coolers or fluidized bed coolers can be used. In the case of fluidized bed apparatus, the heat treatment is effected by means of the hot gas used for the fluidization. The granule coated by the fluidized bed process can be cooled by means of a granule cooler or a fluidized bed cooler similarly to the case of the mixing process. Both in the mixing process and in the fluidized bed process, the coating substance can be sprayed on by means of a one-substance or a two-substance nozzle apparatus. The optional heat treatment consists in heat treatment at a temperature of from 30 to 100° C., but equal to or below the melting or softening temperature of the particular envelope substance. Preference is given to working at a temperature which is just below the melting or softening temperature.

The inventive granular bleach activator mixtures can be used in washing compositions, cleaning compositions and disinfectants together with hydrogen peroxide or inorganic peroxygen compounds. Essential components of such washing compositions, cleaning compositions and disinfectants will be detailed below.

### Surface-Active Substances

### Anionic Surfactants

The washing and cleaning compositions may comprise one or more surfactants, and useful surfactants are in particular anionic surfactants, nonionic surfactants and mixtures thereof, but also cationic, zwitterionic and amphoteric surfactants. Such surfactants are present in the inventive washing compositions in proportions of preferably from 1% by weight to 50% by weight, in particular from 3 to 30% by weight, whereas cleaning compositions for hard surfaces normally contain smaller proportions, i.e. amounts of up to 20% by weight, in particular of up to 10% by weight and preferably in the range from 0.5 to 5% by weight. In cleaning compositions for use in machine dishwashing processes iow-foaming compounds are normally used.

Suitable anionic surfactants are in particular soaps and those which contain sulfate or sulfonate groups. Useful surfactants of the sulfonate type are preferably C₉-C₁₃-alkylbenzenesulfonates, olefinsulfonates, i.e. mixtures of alkene- and hydroxyalkanesulfonates, and also disulfonates, as are obtained, for example, from monoolefins having terminal or internal double bonds by sulfonating with gaseous sulfur trioxide and subsequent alkaline or acidic hydrolysis of the sulfonation products. Also suitable are alkanesulfonates which are obtained from C₁₂-C₁₈-alkanes, for example by sulfochlorination or sulfoxidation with subsequent hydrolysis and neutralization respectively. Also suitable are the esters of alpha-sulfo fatty acids (ester sulfonates) for example the alpha-sulfonated methyl esters of hydrogenated coconut, palm kernel or tallow fat acids, which are prepared by sulfonating the methyl esters of fatty acids of vegetable and/or animal origin having from 8 to 20 carbon atoms in the fatty acid molecule and subsequent neutralization to give water-soluble monosalts.

Further suitable anionic surfactants are sulfated fatty acid glycerol esters which are mono-, di- and triesters, and mixtures thereof. Preferred alk(en)yl sulfates are the alkali metal and in particular the sodium salts of the sulfuric monoesters of the C₁₂-C₁₈-fatty alcohols, for example of coconut fatty alcohol, tallow fatty alcohol, lauryl, myristyl, cetyl or stearyl alcohol, or of the C₈-C₂₀-oxo alcohols and those monoesters of secondary alcohols of this chain length. Also preferred are alk(en)yl sulfates of the chain length mentioned which contain a synthetic, straight-chain alkyl radical prepared on a petrochemical basis. 2,3-Alkyl sulfates, which are prepared, for example, according to U.S. Pat. No. 3,234,158 and U.S. Pat. No. 5,075,041, are also suitable anionic surfactants. Also suitable are the sulfuric monoesters of the straight-chain or branched alcohols ethoxylated with from 1 to 6 mol of ethylene oxide, such as 2-methyl-branched C₉-C₁₁-alcohols with on average 3.5 mol of ethylene oxide (EO) or C₁₂-C₁₈ fatty alcohols having from 1 to 4 EO.

The preferred anionic surfactants also include the salts of alkylsulfosuccinic acid which are also referred to as sulfosuccinates or as sulfosuccinic esters, and the mono- and/or diesters of sulfosuccinic acid with alcohols, preferably with fatty alcohols and in particular with ethoxylated fatty alcohols. Preferred sulfosuccinates contain C₈-C₁₈ fatty alcohol radicals or mixtures of these. Useful further anionic surfactants include fatty acid derivatives of amino acids, for example of N-methyltaurine (taurides) and/or of N-methylglycine (sarcosinates). Useful further anionic surfactants include in particular soaps, for example in amounts of from 0.2 to 5% by weight. Especially suitable are saturated fatty acid soaps, such as the salts of lauric acid, myristic acid, palmitic acid, stearic acid, hydrogenated erucic acid and behenic acid, and also in particular soap mixtures derived from natural fatty acids, for example coconut, palm kernel or tallow fat acids.

The anionic surfactants, including the soaps, may be present in the form of their sodium, potassium or ammonium salts, and as soluble salts of organic bases, such as mono-, di- or triethanolamine. The anionic surfactants are preferably present in the form of their sodium or potassium salts, in particular in the form of the sodium salts. Anionic surfactants are present in inventive washing compositions preferably in amounts of from 0.5 to 10% by weight and in particular in amounts of from 5 to 25% by weight.

### Nonionic Surfactants

The nonionic surfactants used are preferably alkoxylated, advantageously ethoxylated, in particular primary alcohols having preferably from 8 to 18 carbon atoms and on average from 1 to 12 mol of ethylene oxide (EO) per mole of alcohol, in which the alcohol radical may be linear or preferably 2-methyl-branched, or may contain linear and methyl-branched radicals in a mixture, as are typically present in oxoalcohol radicals. However, especially preferred are alcohol ethoxylates having linear radicals from alcohols of native origin having from 12 to 18 carbon atoms, for example from coconut, palm, tallow fat or oleyl alcohol, and on average from 2 to 8 EO per mole of alcohol. Preferred ethoxylated alcohols include, for example, C₁₂-C₁₄-alcohols having 3 EO or 4 EO, C₉-C₁₁-alcohols having 7 EO, C₁₃-C₁₅-alcohols having 3 EO, 5 EO, 7 EO or 8 EO, C₁₂-C₁₈-alcohols having 3 EO, 5 EO or 7 EO and mixtures of these, such as mixtures of C₁₂-C₁₄-alcohol having 3 EO and C₁₂-C₁₈-alcohol having 7 EO. The degrees of ethoxylation specified constitute statistical averages which may be an integer or a fraction for a specific product. Preferred alcohol ethoxylates have a narrow homolog distribution (narrow range ethoxylates, NRE). In addition to these nonionic surfactants, fatty alcohols having more than 12 EO may also be used. Examples thereof are (tallow) fatty alcohols having 14 EO, 16 EO, 20 EO, 25 EO, 30 EO or 40 EO.

The nonionic surfactants also include alkylglycosides of the general formula RO(G)ₓ are used, in which R is a primary, straight-chain or methyl-branched, in particular 2-methyl-branched, aliphatic radical having from 8 to 22, preferably from 12 to 18, carbon atoms, and G is a glycose unit having 5 or 6 carbon atoms, preferably glucose. The degree of oligomerization x which specifies the distribution of monoglycosides and oligoglycosides is an arbitrary number, which may also assume fractional values as a quantity to be determined analytically, between 1 and 10; x is preferably from 1.2 to 1.4. Likewise suitable are polyhydroxy fatty acid amides of the formula (I) in which the R¹CO radical is an aliphatic acyl radical having from 6 to 22 carbon atoms, R² is hydrogen, an alkyl or hydroxyalkyl radical having from 1 to 4 carbon atoms and [Z] is a linear or branched polyhydroxyalkyl radical having from 3 to 10 carbon atoms and from 3 to 10 hydroxyl groups.

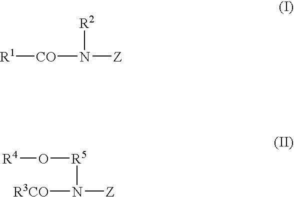

The polyhydroxy fatty acid amides preferably derive from reducing sugars having 5 or 6 carbon atoms, in particular from glucose. The group of the polyhydroxy fatty acid amides also includes compounds of the formula in which (II) is a linear or branched alkyl or alkenyl radical having from 7 to 12 carbon atoms, R⁴ is a linear, branched or cyclic alkylene radical or an arylene radical having from 2 to 8 carbon atoms and R⁵ is a linear, branched or cyclic alkyl radical or an aryl radical, or an oxyalkyl radical having from 1 to 8 carbon atoms, preference being given to C₁-C₄-alkyl or phenyl radicals, and [Z] is a linear polyhydroxyalkyl radical whose alkyl chain is substituted by at least two hydroxyl groups, or alkoxylated, preferably ethoxylated or propoxylated, derivatives of this radical. [Z] is obtained here too preferably by reductive amination of a sugar such as glucose, fructose, maltose, lactose, galactose, mannose or xylose. The N-alkoxy or N-aryloxy-substituted compounds may then be converted to the desired polyhydroxy fatty acid amides by reacting with fatty acid methyl esters in the presence of an alkoxide as a catalyst.

A further class of nonionic surfactants used with preference, which may be used either as the sole nonionic surfactant or in combination with other nonionic surfactants, especially together with alkoxylated fatty alcohols and/or alkylglycosides, is that of alkoxylated, preferably ethoxylated or ethoxylated and propoxylated, fatty acid alkyl esters, preferably having from 1 to 4 carbon atoms in the alkyl chain, in particular fatty acid methyl esters.

Nonionic surfactants of the amine oxide type, for example N-cocoalkyl-N,N-dimethylamine oxide and N-tallowalkyl-N,N-dihydroxyethylamine oxide and of the fatty acid alkanolamides may also be suitable.

Useful further surfactants are what are known as gemini surfactants. This generally refers to those compounds which have two hydrophilic groups per molecule. These groups are generally separated from one another by a “spacer”. This spacer is generally a carbon chain which should be long enough that the hydrophilic groups have a sufficient separation that they can act independently of one another. Such surfactants generally feature an unusually low critical micelle concentration and the ability to greatly reduce the surface tension of water. However, it is also possible to use gemini polyhydroxy fatty acid amides or poly-polyhydroxy fatty acid amides, as described in the international patent applications WO 95/19953, WO 95/19954 and WO 95/19955. Further surfactant types may have dendrimeric structures.

### Builders

### Inorganic Builders

An inventive washing composition preferably comprises at least one water-soluble and/or water-insoluble, organic and/or inorganic builder.

Useful water-soluble inorganic builder materials are in particular alkali metal silicates and polymeric alkali metal phosphates, which may be present in the form of their alkaline, neutral or acidic, sodium or potassium salts. Examples thereof are trisodium phosphate, tetrasodium diphosphate, disodium dihydrogendiphosphate, pentasodium triphosphate, what is known as sodium hexametaphosphate, and the corresponding potassium salts, or mixtures of sodium and potassium salts. The water-insoluble, water-dispersible inorganic builder materials used are in particular crystalline or amorphous alkali metal aluminosilicates, in amounts of up to 50% by weight. Among these, preference is given to the crystalline sodium aluminosilicates in detergent quality, in particular zeolite A, P and optionally X, alone or in mixtures, for example in the form of a cocrystal of zeolites A and X. Their calcium binding capacity is generally in the range from 100 to 200 mg of CaO per gram. Suitable builder substances are also crystalline alkali metal silicates which may be present alone or in a mixture with amorphous silicates. The alkali metal silicates which can be used as builders preferably have a molar ratio of alkali metal oxide to SiO₂ below 0.95, in particular from 1:1.1 to 1:12, and may be present in amorphous or crystalline form. Preferred alkali metal silicates are the sodium silicates, in particular the amorphous sodium silicates having a molar Na₂O:SiO ratio of from 1:2 to 1:2.8. The crystalline silicates used, which may be present alone or in a mixture with amorphous silicates, are preferably crystalline sheet silicates of the general formula Na₂SiₓO₂ₓ₊₁ Y H₂O in which x, known as the modulus, is from 1.9 to 4, and y is from 0 to 20, and preferred values of x are 2, 3 or 4. Preferred crystalline sheet silicates are those in which x in the general formula specified assumes the values of 2 or 3. Preference is given in particular to both β- and β-sodium disilicate (Na₂Si2O₅ y H₂O). It is also possible to use virtually anhydrous, crystalline alkali metal silicates, prepared from amorphous silicates, of the abovementioned general formula, in which x is a number from 1.9 to 2.1. In a further preferred embodiment of such compositions, a crystalline sodium sheet silicate having a modulus of from 2 to 3 is used, as can be prepared from sand and soda. Crystalline sodium silicates having a modulus in the range from 1.9 to 3.5 are used in a further preferred embodiment of inventive compositions. In a preferred embodiment of inventive compositions, a granular compound of alkali metal silicate and alkali metal carbonate is used, as obtainable, for example, under the Nabion® name. If alkali metal aluminosilicate, in particular zeolite, is also present as an additional builder substance, the weight ratio of aluminosilicate to silicate, based in each case on anhydrous active substances, is preferably from 1:10 to 10:1. In compositions which comprise both amorphous and crystalline alkali metal silicates, the weight ratio of amorphous alkali metal silicate to crystalline alkali metal silicate is preferably from 1:2 to 2:1 and in particular from 1:1 to 2:1.

Such builder substances are present in inventive compositions preferably in amounts of up to 60% by weight, in particular from 5 to 40% by weight.

### Organic Builders

The water-soluble organic builder substances include polycarboxylic acids, especially citric acid and sugar acids, aminopolycarboxylic acids, especially methylglycinediacetic acid, nitrilotriacetic acid and ethylenediaminetetra-acetic acid, and also polyaspartic acid.

Polyphosphonic acids, especially aminotris(methylenephosphonic acid), ethylenediaminetetrakis(methylenephosphonic acid) and 1-hydroxyethane-1,1-diphosphonic acid may likewise be used. Preference is also given to polymeric (poly)carboxylic acids, especially the polycarboxylates obtainable by oxidation of polysaccharides and dextrins, polymeric acrylic acids, methacrylic acids, maleic acids and copolymers of these which may also contain small fractions of polymerizable substances without carboxylic acid functionality in copolymerized form. The relative molecular mass of the homopolymers of unsaturated carboxylic acids is generally between 5000 and 200 000, that of the copolymers between 2000 and 200 000, preferably from 50 000 to 120 000, based in each case on free acid. A particularly preferred acrylic acid-maleic acid copolymer has a relative molecular mass of from 50 000 to 100 000. Commercial products are, for example, Sokalan® CP 5, CP 10 and PA 30 from BASF. Also suitable are copolymers of acrylic acid or methacrylic acid with vinyl ethers, such as vinyl methyl ethers, vinyl esters, ethylene, propylene and styrene, in which the proportion of the acid is at least 50% by weight. The water-soluble organic builder substances used may also be terpolymers which contain, as monomers, two unsaturated acids and/or salts thereof and, as a third monomer, vinyl alcohol and/or an esterified vinyl alcohol or a carbohydrate. The first acidic monomer or salt thereof derives from a monoethylenically unsaturated C₃-C₈-carboxylic acid and preferably from a C₃-C₄-monocarboxylic acid, especially from (meth)acrylic acid.

The second acidic monomer or the salt thereof may be a derivative of a C₄-C₈-dicarboxylic acid, particular preference being given to maleic acid, and/or a derivative of an allylsulfonic acid which is substituted in the 2-position by an alkyl or aryl radical. Such polymers generally have a relative molecular mass between 1000 and 200 000. Further preferred copolymers are those which preferably have as monomers acrolein and acrylic acid/acrylic acid salts or vinyl acetate.

Especially for the preparation of liquid compositions, the organic builder substances may be used in the form of aqueous solutions, preferably in the form of from 30 to 50% by weight aqueous solutions. All of the acids mentioned are generally used in the form of their water-soluble salts, especially their alkali metal salts.

Such organic builder substances may, if desired, be present in amounts of up to 40% by weight, in particular up to 25% by weight, and preferably from 1 to 8% by weight. Amounts close to the upper limit mentioned are used preferably in pasty or liquid, especially aqueous compositions.

Useful water-soluble builder components in inventive cleaning compositions for hard surfaces are in principle all builders used customarily in compositions for the machine cleaning of dishes, for example the abovementioned alkali metal phosphates. Their amounts may be in the range of up to about 60% by weight, in particular from 5 to 20% by weight, based on the overall composition. Further possible water-soluble builder components, in addition to polyphosphonates and phosphonate alkyl carboxylates, are, for example, organic polymers of native or synthetic origin of the above-detailed type of the polycarboxylates which function as cobuilders especially in hard water regions, and naturally occurring hydroxycarboxylic acids, for example mono-, dihydroxysuccinic acid, alpha-hydroxypropionic acid and gluconic acid. The organic builder components used with preference include the salts of citric acid, especially sodium citrate. The sodium citrate used is anhydrous trisodium citrate and preferably trisodium citrate dihydrate. Trisodium citrate dihydrate may be used in the form of finely or coarsely crystalline powder. Depending upon the pH set ultimately in the inventive cleaning compositions, the acids corresponding to the cobuilder salts mentioned may also be present.

### Enzymes

The enzymes optionally present in the inventive compositions include proteases, amylases, pullulanases, cellulases, cutinases and/or lipases, for example proteases such as BLAP®, Optimas®, Opticlean®, Maxacal®, Maxapem®, Durazym®, Purafect® OxP, Esperase® and/or Savinase®, amylases such as Termamy®, Amylase-LT, Maxamyl®, Duramyl®, Purafectel OxAm, cellulases such as Celluzyme®, Carezyme®, K-AC® and/or lipases, such as Lipolase®, Lipomax®, Lumafast® and/or Lipozym®. The enzymes used may be adsorbed on carriers and/or embedded in envelope substances in order to protect them from premature inactivation. They are present in inventive washing and cleaning compositions preferably in amounts of up to 10% by weight, in particular from 0.05 to 5% by weight, particular preference being given to the use of enzymes stabilized against oxidative degradation.

Inventive machine dishwasher detergents preferably comprise the customary alkali carriers, for example alkali metal silicates, alkali metal carbonates and/or alkali metal hydrogencarbonates. The customarily used alkali carriers include carbonates, hydrogencarbonates and alkali metal silicates having a molar SiO₂/M₂O ratio (M=alkali metal atom) of from 1:1 to 2.5:1. Alkali metal silicates may be present in amounts of up to 40% by weight, in particular from 3 to 30% by weight, based on the overall composition. The alkali carrier system used with preference in the inventive cleaning compositions is a mixture of carbonate and hydrogencarbonate, preferably sodium carbonate and hydrogencarbonate which may be present in an amount of up to 50% by weight, preferably from 5 to 40% by weight.

In a further embodiment of inventive compositions for the automatic washing of dishes, from 20 to 60% by weight of water-soluble organic builders, in particular alkali metal citrate, from 3 to 20% by weight of alkali metal carbonate and from 3 to 40% by weight of alkali metal disilicate are present.

In order to bring about silver corrosion protection, it is possible to use silver corrosion inhibitors in inventive cleaning compositions for dishes. Preferred silver corrosion protectants are organic sulfides such as cystine and cysteine, di- or trihydric phenols, optionally alkyl- or aryl-substituted triazoles such as benzotriazole, isocyanuric acid, salts and/or complexes of titanium, zirconium, hafnium, molybdenum, vanadium or cerium, and salts and/or complexes of the metals present in complexes suitable in accordance with the invention with ligands other than those specified in formula (I).

When the compositions foam too vigorously on use, it is possible also to add to them up to 6% by weight, preferably from about 0.5 to 4% by weight, of a foam-regulating compound, preferably from the group comprising silicones, paraffins, paraffin-alcohol combinations, hydrophobicized silicas, fatty acid bisamides and mixtures thereof, and other known commercially available foam inhibitors. The foam inhibitors, especially silicone- and/or paraffin-containing foam inhibitors, are preferably bound to a granular carrier substance soluble or dispersible in water. Special preference is given to mixtures of paraffins and bistearylethylenediamide. Further optional ingredients in the inventive compositions are, for example, perfume oils.

To set a desired pH which does not arise automatically by the mixing of the remaining components, the inventive compositions may comprise system- and environment-compatible acids, especially citric acid, acetic acid, tartaric acid, malic acid, lactic acid, glycolic acid, succinic acid, glutaric acid and/or adipic acid, but also mineral acids, especially sulfuric acid or alkali metal hydrogensulfates, or bases, especially ammonium or alkali metal hydroxides. Such pH regulators are present in the inventive compositions preferably to the extent of not more than 10% by weight, in particular from 0.5 to 6% by weight.

To produce particulate compositions with increased bulk density, especially in the range from 650 g/l to 950 g/l, preference is given to a process which has an extrusion step and is disclosed by the European patent EP 0 486 592. A further preferred production method with the aid of a granulation process is described in the European patent EP 0 642 576. Inventive compositions in the form of nondusting, storage-stably free-flowing powders and/or granules having high bulk densities in the range from 800 to 1000 g/l can also be prepared by mixing, in a first process stage, the builder components with at least a portion of liquid mixture components while increasing the bulk density of this premixture, and subsequently, if desired after an intermediate drying, combining the further constituents of the composition, including the bleach catalyst, with the thus obtained premixture.

To prepare the inventive compositions in tablet form, the procedure is preferably to mix all constituents with one another in a mixer and to compress the mixture by means of conventional tablet presses, for example eccentric presses or rotary presses, with compression pressures in the range from 200 10⁵ Pa to 1500 10⁵ Pa. In this way, tablets which are fracture-resistant and nevertheless sufficiently rapidly soluble under use conditions and have flexural strengths of normally above 150 N are obtained without any problem. A tablet prepared in this way preferably has a weight of from 1-5 g to 40 g, in particular from 20 g to 30 g, at a diameter of from 3-5 mm to 40 mm.

### EXAMPLES

### Example 1

### Pelletization of a Cogranule Composed of Tetraacetylethylenediamine (TAED) and Nonanoyloxybenzoic Acid (DOBA)

In a customary laboratory mixer (e.g. Lödige M5R), 221 g of TAED powder and 310 g of DOBA were mixed, using DOBA as a moist filtercake with a residual moisture content of approx. 28.8%. The homogenized mixture was subsequently metered into an edge-runner press (PP 85 pelletizing press from Schlüter), which was equipped with a ring die with 1 mm bores. At a die speed of n=300 min⁻¹, an amount of 491 g of mixture was granulated within 3 min to pellets having a diameter of D=1 mm of length of approx. 0.75-3.5 mm. The pellets were dried in a laboratory fluidized bed dryer (from Retsch) at an air entrance temperature of T=70° C. for 20 min. The final screening-out of the granules gave rise to a yield of 83.7% in the target fraction of 400-1600 μm; the fine fraction <400 μm was 16.3%; there was no coarse fraction >1600 μm.

### Example 2

### Granulation of a Cogranule Composed of TAED and DOBA

In a laboratory mixer, 56.4 g of TAED powder and 81.5 g of DOBA were mixed, using DOBA as a moist filtercake having a residual moisture content of approx. 30.5%. 21.1 g of water were also added to the mixture. The homogenized mixture was subsequently metered into a dome extruder (DG-L1 from Fuji-Paudal), which was equipped with a die having 1 mm bores. At an extruder speed of n=45 min⁻¹, an amount of 147 g of mixture was processed to extrudates within 1 min. The extrudates were rounded to spherical granules directly thereafter in a rounder (BR 300 from Hosokawa-Bepex) at a speed of n=600 min⁻¹. The granules were dried in a laboratory fluidized bed dryer (from Retsch) at an air entrance temperature of T=70° C. for 20 min. The final screening-out of the granules gave rise to a yield of 81.6% in the target fraction of 400-1600 μm; the fine fraction <400 μm was 5.6%; the coarse fraction >1600 μm was 12.8%.

1. A granular bleach activator mixture consisting essentially of
a) a hydroxybenzoic acid derivative of the formula 1 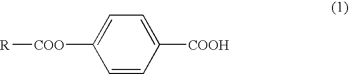
in which R is C₈-C₁₁-alkyl, and
b) tetraacetylethylenediamine or 1,5-diacetyl-2,4-dioxo-1,3,5-hexahydrotriazine, or a mixture thereof, said granular bleach activator mixture being free of binders.

2. The granular bleach activator mixture as claimed in claim 1, consisting of from 5 to 95% by weight of the hydroxybenzoic acid derivative of the formula 1 and from 95 to 5% by weight of tetraacetylethylenediamine or 1,5-diacetyl-2,4-dioxo-1,3,5-hexahydrotriazine, or a mixture thereof.

3. The granular bleach activator mixture as claimed in claim 1, consisting of from 25 to 75% by weight of the hydroxybenzoic acid derivative of the formula 1 and from 75 to 25% by weight of tetraacetylethylenediamine or 15-diacetyl-2,4-dioxo-1,3,5-hexahydrotriazine or a mixture thereof.

4. The granular bleach activator mixture as claimed in claim 1, consisting of from 60 to 40% by weight of the hydroxybenzoic acid derivative of the formula 1 and from 40 to 60% by weight of tetraacetylethylenediamine or 1,5-diacetyl-2,4-dioxo-1,3,5-hexahydrotriazine or a mixture thereof.

5. The granular bleach activator mixture as claimed in claim 1, consisting of from 95 to 100% by weight of the bleach activators a) and b) and water to 100% by weight.

6. The granular bleach activator mixture as claimed in claim 1, which is present in finished form.

7. The bleach activator mixture as claimed in claim 1, which additionally comprises additives.

8. A washing, cleaning or disinfectant composition comprising a granular bleach activator mixture as claimed in claim 1 and hydrogen peroxide or an inorganic peroxygen compound.
